(19) United States
(12) Patent Application Publication
(10) Pub. No.: US 2004/0220409 A1
(43) Pub. Date: Nov. 4, 2004

(54) Flecainide synthesis

(76) Inventors: William C. McDaniel, Grove Village, IL (US); Jayaramaiyer Radhakrishnan, Westchester, IL (US); Slawomir J. Janicki, North Chelmsford, MA (US)

Correspondence Address: DARBY & DARBY P.C., P. O. BOX 5257, NEW YORK, NY, 10150-5257, US

(21) Appl. No.: 10/468,628

(22) PCT Filed: Feb. 20, 2002
(86) PCT No.: PCT/US02/05390

Related U.S. Application Data
(60) Provisional application No. 60/270,048, filed on Feb. 20, 2001.
(60) Provisional application No. 60/271,788, filed on Feb. 27, 2001.

(51) Int. Cl. C07D 211/32

(52) U.S. Cl. 546/233

(57) ABSTRACT

An improved, highly efficient method for the preparation of flecainide acetate (I) or other pharmaceutically acceptable salts of flecainide. The method involves a technique for preparing the staring material 1,4-bis (2,2,2-trifuoroethoxy) benzene (III) in high yields by reacting 4-fluoro-1-bromobenzene (VI) with TFE in the presence of a base and a copper-containing catalyst.

This application claims the benefit of priority under 35 U.S.C. § 119 based upon U.S. Ser. No. 60/270,048, filed Feb. 20, 2001, and U.S. Ser. No. 60/271,788, filed Feb. 27, 2001, the entire disclosures of which are incorporated herein by reference.

### FIELD OF THE INVENTION

The present invention describes an improvement in the method of preparing flecainide from 1,4-bis(2,2,2-trifluoroethoxy)benzene.

### BACKGROUND OF THE INVENTION

Flecainide acetate (I), 2,5-bis(2,2,2-trifluoroethoxy)-N-(2-piperidylmethyl)-benzamide acetate, having the formula:

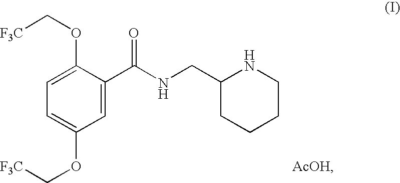

is an effective antiarrythmic drug. Flecainide produces its antiarrythmic effect by decreasing nerve impulses in the heart. This effect is proposed to be due to inhibition of sodium and potassium channels present on the heart (International Patent Application Nos. PCT/IL98/00315 and WO 99/02498; U.S. Pat. No. 3,900,481). Flecainide also has been shown to decrease heart rate.

One prior art method for preparing flecainide, disclosed in British Published Patent Application GB 2,045,760, starts from 2,5-bis(2,2,2-trifluoroethoxy) benzoic acid:

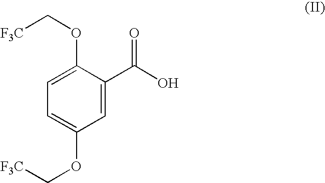

This starting material is prepared by a multistage process, comprising the conversion of 1,4-dibromobenzene to 1,4-bis(2,2,2-trifluoroethoxy) benzene:

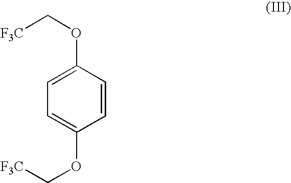

by reacting 8 equivalents of 2,2,2-trifluoroethanol (TFE) per mole of the dibromobenzene, and then acetylating the (trifluoroethoxy)benzene to form 2,5-bis(2,2,2-trifluoroethoxy) acetophenone. Alternatively, hydroquinone can be converted to 1,4-bis(2,2,2-trifluoroethoxy) benzene (m) using trifluoroethyl triflate (CF₃CH₂OSO₂CF₃). The acetophenone then is oxidized to form the corresponding benzoic acid (II).

The benzoic acid then is converted to its acid chloride and reacted with 2-(aminomethyl)-piperidine to form flecainide in one step. Alternatively, the acid chloride can be reacted with 2-(aminomethyl)pyridine, followed by catalytic hydrogenation of the pyridine ring to form flecainide.

A serious disadvantage of using 1,4-dibromobenzene to form 1,4-bis(2,2,2-trifluoroethoxy) benzene (m) is that the process requires the reaction of 8 equivalents of TFE while only 2 equivalents are theoretically needed. The use of less than 8 equivalents of TFE results in incomplete conversion to 1,4-bis(2,2,2,-trifluoroethoxy)benzene, with the starting material and 1-bromo-4-(2,2,2-trifluoroethoxy) benzene as the main impurities. Isolation and purification of the desired product from this mixture is not practical on an industrial scale. A further disadvantage is that the acid chloride intermediate disclosed in GB 2,045,760 is a liquid, which cannot be stored for prolonged periods of time, but must be used immediately after it is prepared. The one step condensation process with 2-(aminomethyl)-piperidine also results in a mixture of two acylated isomers since the acid chloride reacts non-selectively with both nitrogen atoms of the piperidine moiety. For this reason, the two-step process via the pyridine intermediate is commercially preferred over the one-step process. In the two-step process, however, flecainide acetate is isolated with a low total yield of about 71%.

U.S. Pat. No. 3,655,728, later reported in J. Med. Chem. 1975, 18, 1130; disclosed formation of methyl 2,5-bis(2,2,2-trifluoroethoxy)benzoate and other trifluoroethyl trifluoroethoxyaryl esters from methyl gentisate.

U.S. Pat. No. 3,900,481, reported in J. Med. Chem. 1977, 20, 821; described the conversion of 2,2,2-trifluoroethyl 2,5-bis(2,2,2-trifluoroethoxy) benzoate:

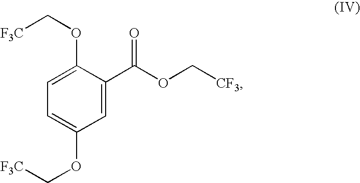

to flecainide by two synthetic routes. Thus, the (2,2,2-trifluoroethoxy)benzoate may be reacted with 2-aminomethylpiperidine to produce the final compound. Alternatively, the (2,2,2-trifluoroethoxy)benzoate may be reacted with 2-aminomethylpyridine, and thereafter reduced in the presence of hydrogen chloride to form flecainide hydrochloride. The acid chloride also may be reacted with 2-aminomethylpyridine and then reduced to produce the desired compound. This method, however, suffers the same disadvantages as discussed for the process disclosed in GB 2,045,760. Another disadvantage of this method is that the crude acid chloride intermediate is too impure to be used without purification and purification thereof requires high vacuum distillation and results in considerable decomposition of the intermediate.

U.S. Pat. No. 3,419,595 describes the formation of triflate, CF₂CH₃OSO₂CF₃, and the subsequent reaction of that compound with phenols to form 2,2,2-trifluoroethoxyaryl compounds.

U.S. Pat. No. 4,005,209 describes the reaction of 2,2,2-trifluoroethyl trifluorosulfonate, 2,5-bis(2,2,2-trifluoroethoxy) benzoate (IV) with 2-aminomethylpyridine and the reduction of other analogous pyridinebenzamide derivatives to produce flecainide. Two serious disadvantages of this method are (1) 2,2,2-trifluoroethyl trifluorosulfonate is expensive and (2) preparation of 2,2,2-trifluoroethyl trifluorosulfonate requires the use of halogenated trifluorosulfonate (CF₃SO₂X, where x=F or Cl), which is highly corrosive and toxic.

U.S. Pat. No. 4,642,384 describes the formation of 1,4-bis(2,2,2-trifluoroethoxy) benzene (E) from hydroquinone, K₂CO₃ and trifluoroethyl triflate. 1,4-bis(2,2,2-trifluoroethoxy) benzene (m) is formed by reaction of 1,4-dibromobenzene and the sodium salt of TFE in the presence of DMF and a copper salt catalyst. The product then may be acetylated with Ac₂O or AcCl in the presence of a Lewis acid to form the 2,5-bis(2,2,2-trifluoroethoxy) acetophenone, which is chlorinated to form the 2,5-bis(2,2,2-trifluoroethoxy) trichloroacetophenone. The latter compound is condensed with 2-aminomethylpyridine to form the pyrdinyl benzamide (V) which is reduced to Flecainide acetate (I). The benzamide (V) has the structure:

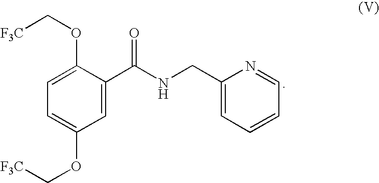

Alternatively, the 2,5-bis(2,2,2-trifluoroethoxy) trichloroacetophenone is condensed with 2-aminomethylpiperidine, to give flecainide acetate directly. The one step process has a serious disadvantage in that the 2,5-bis(2,2,2-trifluoroethoxy) trichloroacetophenone can react non-selectively with either nitrogen of the 2-aminomethylpiperidine to produce a mixture of two acylated isomers.

U.S. Pat. No. 4,642,384 describes isolation of the flecainide acetate by filtration of the final reaction mixture to remove the catalyst, evaporation of the solvent to dryness and trituration of the semisolid residue with hexane followed by filtration of the slurry to obtain the crude product as a solid. Recrystalization of the crude product afforded pure flecainide acetate. Isolation of the crude product by this process is impractical on a commercial scale, since the complete removal of solvent by evaporation is difficult and the semisolid residue is intractable in large scale operations.

PCT Publication Nos. WO 99/02498 and 98/47853 disclose the formation of 2,5-bis(2,2,2-trifluoroethoxy) benzoic acid (II) by alkoxylation of expensive halobenzoic acid or a salt, wherein the benzoic acid may be substituted with either Cl, Br, I, or CF₃CH₂—. The reagents used are 7.8 equivalents of TFE, a suitable polar solvent, a strong base, and copper bromide and copper iodide. The resulting 2,5-bis(2,2,2-trifluoroethoxy) benzoic acid (II) is then activated using haloacetonitrile to produce an intermediate ester. The ester is reacted with 2-(aminomethyl) piperidine or 2-(aminomethyl) pyridine to produce the amide or flecainide, respectively. The amide then can be converted to flecainide by catalytic hydrogenation. There are several disadvantages of this reaction scheme including, but not limited to, (1) the high cost of the initial halobenzoic acid; (2) the use of 7.8 equivalents of expensive TFE in the first step while only 2 equivalents are theoretically required; (3) poor yields of the alkoxylation reaction to produce 2,5-bis(2,2,2-trifluoroethoxy) benzoic acid; and (4) the high cost and high toxicity of the required haloacetonitrile.

Accordingly, it may be seen that there is a continuing need to develop improved reaction schemes for producing flecainide in safer and less expensive ways.

### SUMMARY OF THE INVENTION

The present invention relates to an improved, highly efficient method for the preparation of flecainide acetate (I) or other pharmaceutically acceptable salts of flecainide. An important feature of the method of the invention is an improved technique for preparing the starting material 1,4-bis (2,2,2-trifluoroethoxy) benzene (III) in high yields, e.g., of from about 60 to 70%, by reacting 4-fluoro-1-bromobenzene (VI) with TFE:

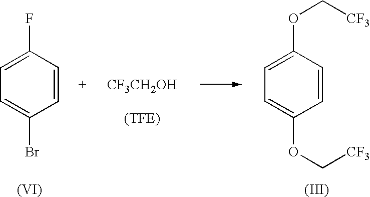

in the presence of a strong base and a copper-containing catalyst, the TFE preferably being reacted in the proportion of about 1 to 3 moles per mole of the 4-fluoro-1-bromobenzene. An advantage of the use of this initial step for forming the (trifluoroethoxy) benzene starting material is that it requires only about 2 equivalents of TFE, whereas prior art procedures for forming that compound require about 8 equivalents. As pointed out above, the use of less than 8 equivalents of TFE in the prior art reactions results in incomplete conversion to the 1,4-bis (2,2,2,-trifluoroethoxy) benzene, with the starting material and 1-bromo-4-(2,2,2-trifluoroethoxy) benzene as the main impurities. Isolation and purification of the desired product from this mixture is not practical on an industrial scale. The reaction of 4-fluoro-1-bromobenzene with TFE avoids the need for significant purification of the product from the reaction mixture.

The (trifluoroethoxy) benzene (III) is then converted to 2,5-bis (2,2,2-trifluoroethoxy) benzoyl halide (VII), and the latter is reacted with TFE in the presence of an appropriate base to produce 2,5-bis (2,2,2-trifluoroetboxy) benzoate (IV). Finally, the (trifluoroethoxy) benzoate is reacted with 2-aminomethylpyridine to form 2,5-bis (2,2,2-trifluoroethoxy pyridylbenzamide (V), and the latter is reduced to flecainide and purified.

The flecanide may then be reacted with acetic acid to produce flecainide acetate (I). The mixture then is concentrated to produce a concentrated reaction mixture and diluted with an antisolvent for the flecainide acetate. The flecainide acetate (I) then can be isolated from the mixture.

Further in accordance with the invention, the (trifluoroethoxy) benzene (III) may be converted to the (trifluoroethoxy) benzoyl halide (VII) by either of two techniques. In a first, preferred reaction sequence the (trifluoroethoxy) benezene is reacted with a chlorooxoacetate ROC(O)COCl in the presence of a suitable Lewis acid catalyst to produce a corresponding alkyl 2,5-bis (2,2,2-trifluoroethoxy) phenylglyoxalate (VIII), and the latter is oxidized to produce the 2,5-bis (2,2,2-trifluoroethoxy) benzoic acid (II) in nearly quantitative yields. The (trifluoroethoxy) benzoyl chloride (VII) is then produced by reacting the (trifluoroethoxy) benzoic acid with a suitable acyl chloride. Preferably, the alkyl 2,5-bis(2,2,2-trifluoroethoxy) phenylglyoxalate (VIII) is methyl 2,5-bis(2,2,2-trifluoroethoxy) phenylglyoxalate (VIIIa). The Lewis acid catalyst is suitably aluminum trichloride or boron trifluoride.

Alternatively, the (trifluoroethoxy) benzoyl halide (VII) intermediate is produced by catalytic reaction of the (trifluoroethoxy) benzene (III) with an oxalyl halide, the (trifluoroethoxy) benzoyl halide being formed in admixture with 2,5-bis(2,2,2-trifluoroethoxy benzoic acid anhydride (XI), 2,5-bis(2,2,2-trifluoroethoxy) benzoic acid (II) and bis(2,5-bis(2,2,2-trifluoroethoxy) phenyl ketone (XII). The (trifluoroethoxy) benzoyl halide (VII) optionally may then be separated from the mixture.

### DETAILED DESCRIPTION OF THE INVENTION

As indicated above, the method of the present invention involves the initial preparation of 1,4-bis (2,2,2-trifluoroethoxy) benzene (III) by reacting 4-fluoro-1-bromobenzene (VI) with TFE in the presence of a strong base and a copper-containing catalyst. The TFE is reacted in the proportion of about 1-3 moles, preferably about 2-2.5 moles, per mole of the 4-fluoro-1-bromobenzene to displace both halogens thereon.

Bases in which the reaction may be carried out include alkali metal or alkaline earth metal hydrides, e.g. sodium hydride (NaH), lithium hydride (LiH), or calcium hydride (CaH₂); alkali metal or alkaline earth metal hydroxides, e.g., sodium hydroxide (NaOH); and calcium carbonate (CaCO₃). Copper-containing materials useful as catalysts in the reaction include copper salts, e.g., copper bromide (CuBr₂); oxides, e.g., CuO and CuO₂; alloys, e.g., Cu—Zn; and metallic copper. The reaction may be performed in any appropriate solvent, preferably an aprotic solvent. Examples of aprotic solvents so useful include, but are not limited to, N,N-dimethylformnamide, N-methylpyrrolidone, N,N-dimethylacetamide, and N,N,N′,N′-tetramethylurea. The reaction may be carried out at temperatures from ambient to about 140° C., preferably at about 100 to 105° C., most desirably at about 100° to 102° C. In accordance with a particularly preferred embodiment, the 4-fluoro-1-bromobenzene (VI) is reacted with sodium 2,2,2-trifluoroethanolate (generated from reacting TFE and NaH) in N,N-dimethylformamide in the presence of CuBr₂ at about 100° C.-105° C.

The following flow sheets illustrate the subsequent steps of the method of the invention, commencing with conversion of the (trifluoroethoxy) benzene (III) to 2,2,2-trifluoroethyl 2,5-bis (2,2,2-trifluoroethoxy) benzoate (IV) shown in Reaction Scheme A, followed by conversion of the latter to flecainide acetate as shown in Reaction Scheme B. Reaction Scheme A further illustrates the preferred sequence a for sequentially converting the (trifluoroethoxy) benzene to the corresponding phenylglyoxalate (VIII), the benzoic acid (II) and the benzoyl chloride (VII) derivatives (Stage 2a), and forming the 2,2,2-trifluoroethyl benzoate derivative (IV) therefrom (Stage 3); and the alternative sequence b, for converting the (trifluoroethoxy) benzene directly to the benzoyl chloride (VII) derivative (Stage 2b) and, after purification, converting that compound to the benzoate (IV):

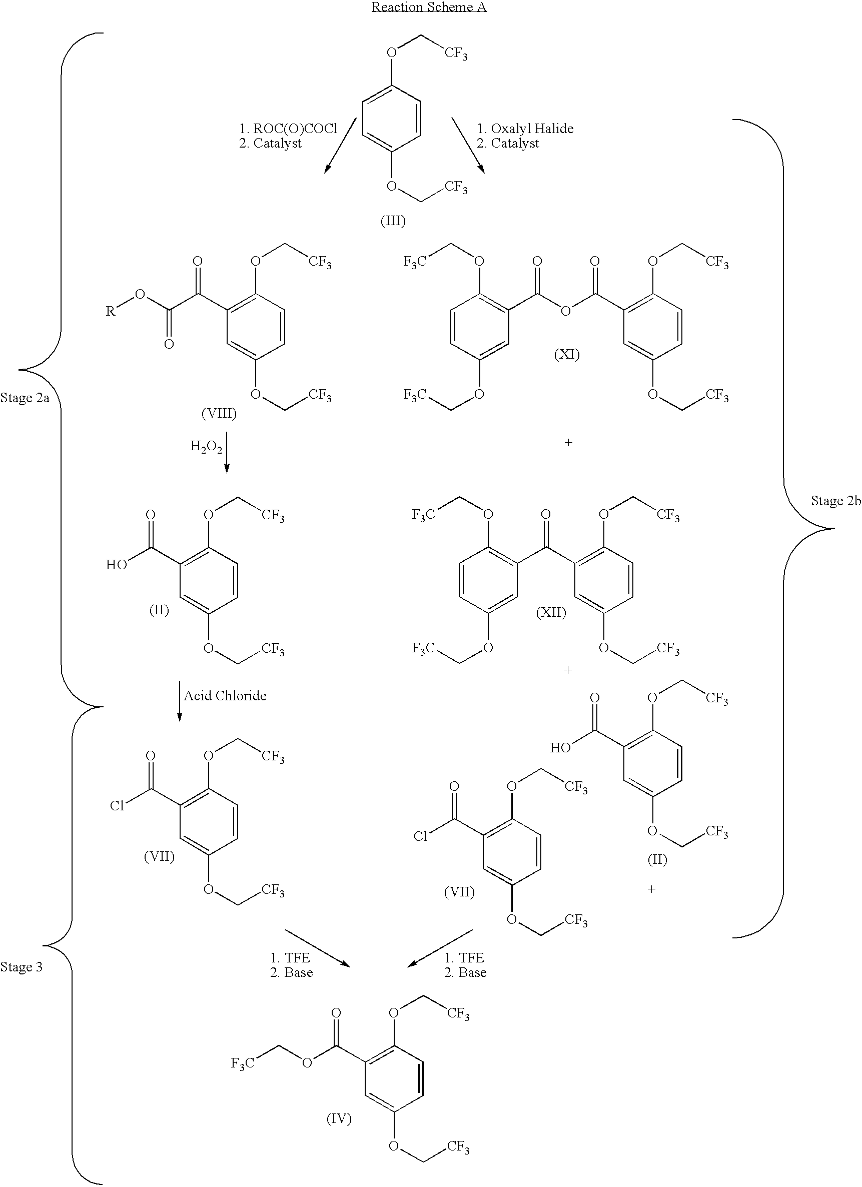

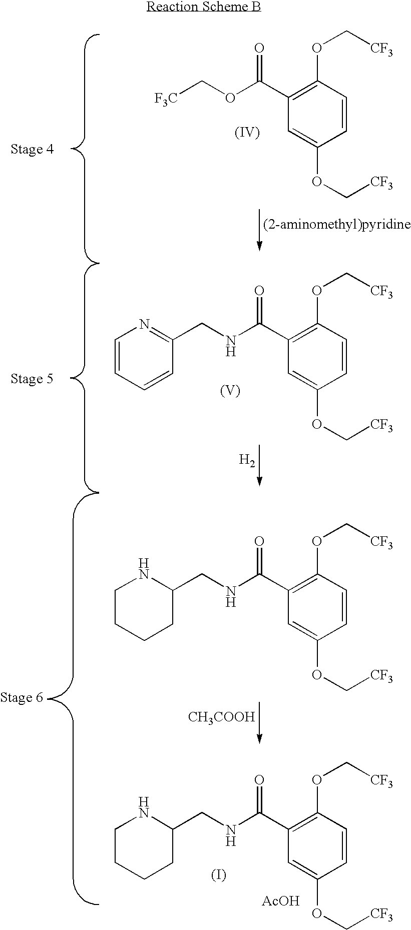

a. Conversion of the (Trifluoroethoxy) Benzene Through the Phenylglyoxalate to the Benzoyl Halide (VII)

As illustrated in Reaction Scheme A, Stage 2a, the (trifluoroethoxy) benzene (III) is reacted with a chlorooxoacetate ROC(O)COCl, wherein R is C₁-C₆ alkyl, preferably methyl or ethyl, in the presence of a suitable catalyst. Examples of suitable catalysts include, but are not limited to, aluminum trichloride, boron trifluoride, and other Lewis acid catalysts. The respective reactants are present in a molar ratio of about 1:4, preferably about 0.40 to 0.81. The reaction is performed at temperatures of about 15 to 30° C., preferably about 20 to 25° C. Particular reaction conditions may be selected by analogy to other Friedel-Crafts acylations of alkoxybenzenes. (See, for example, Olah, “Friedel-Crafts and Related Reaction”, Interscience, New York, 1963-1964 and Sannicolo, Gaza. Chim. Ital. 1985, 115:91).

In a particularly preferred embodiment of the present invention, 1,4-bis(2,2,2-trifluoroethoxy) benzene (E) is reacted with methyl chlorooxoacetate and aluminum chloride in methylene chloride at about 20-25° C. to yield methyl 2,5-bis(2,2,2-trifluoroethoxy) phenylglyoxalate (VIIIa).

The (trifluoroethoxy) phenylglyoxalate (VIII) is thereafter oxidized in the presence of a suitable oxidant and base to produce 2,5-bis(2,2,2-trifluoroethoxy) benzoic acid (II). Particular reaction conditions may be selected by analogy to other oxidations described in the literature. (See, for example, Syn. Comm., 1989, 19:2987).

In the practice of the method of this invention, the trifluoroethoxy benzoic acid (II) is produced in nearly quantitative yields, utilizing the reactants in a molar ratio of about 1:4, 2,5-bis(2,2,2-trifluoroethoxy) phenylglyoxalate:oxidant, preferably in a ratio of about 1:1.4. The reaction may be performed at temperatures of about 0° to 70° C., preferably about 38° to 42° C. Examples of suitable oxidants include but are not limited to, NaOCl, KMnO₄, and hydrogen peroxide (H₂O₂) or H₂O₂-generating substances (e.g., metal peroxides, metal percarbonates, and metal perborates); preferably H₂O₂. Examples of bases include, but are not limited to, alkali metal, alkaline earth metal, or ammonium hydroxides, e.g., NaOH, KOH, LiOH, Ca(OH)₂, and Mg(OH)₂; preferably NaOH; or quaternary ammonium bases, e.g., n-Bu₄NOH.

In a particularly preferred embodiment, methyl 2,5-bis(2,2,2-trifluoroethoxy) phenylglyoxalate (VIIIa) is oxidized by hydrogen peroxide in the presence of NaOH to yield 2,5-bis(2,2,2-trifluoroethoxy) benzoic acid (II).

In the final step of Stage 2a, the (trifluoroethoxy) benzoic acid is converted to the corresponding benzoyl halide, preferably the benzoylchloride (VII) by reaction with a suitable acid halide, preferably an acid chloride such as thionyl chloride, phosphorus trichloride, phosphorus pentachloride, phosphorus oxychloride, phosgene or oxalyl chloride. The reaction may be carried out as more fully described in GB 2,045,760.

b. Direct Conversion of the (Trifluoroethoxy) Benzene to Mixtures Containing Benzoyl Halide (VIII)

As noted above, in the alternative reaction sequence for forming the (trifluoroethoxy) benzoyl chloride (VII), illustrated as Stage 2b in Reaction Scheme A, the 1,4-bis(2,2,2-trifluoroethoxy) benzene (III) is reacted with an oxalyl halide in the presence of a catalyst to produce a mixture of 2,5-bis(2,2,2-trifluoroethoxy) benzoic acid anhydride (XI), 2,5-bis(2,2,2-trifluoroethoxy) benzoic acid (II), bis(2,5-bis(2,2,2-trifluoroethoxy) phenyl) ketone (XII), and the 2,5-bis(2,2,2-trifluoroethoxy) benzoyl halide, preferably the benzoyl chloride (VII). Examples of oxalyl halides that may be so utilized include oxalyl chloride and oxalyl bromide. Catalysts so useful include aluminum trichloride, boron trifluoride, and other Lewis acid catalysts. The reactants are preferably reacted in a molar ratio of about 1:10, 1,4-bis(2,2,2-trifluoroethoxy) benzene:oxalyl halide, preferably in a ratio of about 1:8. The reaction may be performed at temperatures of about 0 to 35° C., preferably about 20° to 25° C.

The benzoyl chloride (VII), prepared as described in either of Stages 2a or 2b can be used without further purification or may be separated from the other reaction products formed in Stage 2b, and purified (in the case of both Stages 2a and 2b), by conventional means. Examples of methods that may be used to separate and purify the benzoyl chloride from the other reaction products include, but are not limited to, vacuum distillation and extraction with organic solvents, e.g., toluene or methylene chloride.

c. Conversion of the Benzoyl Halide (VII) To the Benzoate (IV)

The benzoyl chloride, prepared as described in connection with either of sequences a, or b, above, is reacted (Stage 3 in Reaction Scheme A) with TFE in an inert solvent and in the presence of an appropriate base to form 2,2,2-trifluoroethyl 2,5-bis(2,2,2-trifluoroethoxy) benzoate (IV). Suitable bases include, but are not limited to, tertiary aliphatic amines, whether branched or unbranched, e.g., amines of the formula N(CₙH₂ₙ₊₁)₃, where n is 1 to 7, triethylamine being particularly preferred; and heterocyclic amines, whether saturated or unsaturated. Suitable inert solvents include aromatic hydrocarbons, e.g., toluene and xylenes; aliphatic hydrocarbons, whether branched or unbranched e.g., hexane, heptane, and octane; alicyclic hydrocarbons, mono- and polycyclic, e.g., cyclohexane, tetraline, and decalin. A preferred solvent is toluene. The reactants are reacted in a molar ratio of about 1:4, 2,5-bis(2,2,2-trifluoroethoxy) benzoyl chloride:base; preferably, the ratio is about 1:2. The reaction may be performed at temperatures of about 70° to 120° C., preferably at about 90° to 105° C.

d. Conversion of the Benzoate (IV) To Flecainide Acetate (I)

The 2,2,2-trifluoroethyl 2,5-bis(2,2,2-trifluoroethoxy) benzoate (IV) is reacted with (2-aminomethyl) pyridine to yield the pyridylbenzamide (V) (Stage 4 in Reaction Scheme B). The reaction may be carried out as more fully described in U.S. Pat. No. 4,005,209.

In Stage 5 of Reaction Scheme B, the pyridylbenzamide (V) is hydrogenated in the presence of a platinum oxide or platinum on carbon catalyst in a suitable solvent, e.g., acetic acid. The reduction can be performed under hydrogen pressures of about 5 to 55 psi preferably about 10 psi. The reaction may be carried out in about 1 to 20 hours, preferably about 3 hours.

The residual filtrate is reacted with a suitable base to produce the flecainide free base; Stage 5 of Reaction Scheme B. Suitable bases include, but are not limited to, alkali metal and alkaline earth metal hydroxides, e.g., NaOH, or KOH; and alkali metal or alkaline earth metal hydroxides, carbonates, or bicarbonates; NaOH being particularly preferred. The bases may be in solid or aqueous form. The reaction may be carried out at about 0° to 25° C., preferably at about 5° to 10° C. In a preferred embodiment, the filtrate is cooled to about 5° to 10° C. and reacted with 50% aqueous NaOH for about 2 hours.

The flecainide free base then is converted to flecainide acetate (I), as shown in Stage 6 of Reaction Scheme B. The free base is thus reacted with acetic acid to form the acetate. The reaction may be performed at ambient temperatures, preferably about 20° to 25° C. The reaction may be carried out in about 1 to 10 hours, preferably about 2 hours.

The flecainide acetate slurry is concentrated and then diluted with a suitable antisolvent, e.g., hexane or another branched or unbranched aliphatic hydrocarbon; or an alicyclic hydrocarbon, whether mono- or polycyclic, e.g., cyclohexane, tetraline, or decalin; hexane being particularly preferred.

### EXAMPLES

The present invention will be better understood by reference to the following Examples, which are provided as exemplary of the invention, and not by way of limitation.

### Example 1:Preparation of Flecainide Acetate

(a) Preparation of 1,4-bis(2,2,2-trifluoroethoxy) Benzene (III):

To a stirred mixture of anhydrous dimethylformamide (12 mL) and NaOH (60% mineral oil dispersion, 2.7 g, 0.06742 mole) at 0°-5° C. was added 2,2,2-trifluoroethanol (TFE) (2.29 g, 0.02286 mole) dropwise over 15 minutes under an atmosphere of dry nitrogen. After the addition was complete the slurry was warmed up and stirred at 20° C.-25° C. for 1 hour.

Cupric bromide (0.36 g, 0.0016 mole) and 1-bromo-4-fluorobenzene (2.0 g, 0.001143 mole) were then added in sequence at 20° C.-25° C. The resulting slurry was heated and stirred at 100° C.-102° C. for a total of 18 hours.

The reaction mixture was cooled to 20° C.-25° C., quenched onto 100 mL of water, and acidified to pH=1 using 10% aqueous HCl with stirring. The product was extracted with dichloromethane (2×25 mL) and then isolated by evaporation of solvents. The product was a mixture of unreacted starting material (16.3%), 4-fluoro-1-(2,2,2-trifluoroethoxy) benzene (21.1%) and 1,4-bis(2,2,2-trifluoroethoxy) benzene (62.5%).

### Reaction Scheme A, Stages 2a and 3

(b) Preparation of Alkyl 2,5-bis(2,2,2-trifluoroethoxy) Phenylglyoxalate (VIII):

(1) Preparation of Methyl 2,5-bis(2,2,2,-trifluoroethoxy) Phenylglyoxalate (VIIIa)

To a stirred slurry of anhydrous aluminum chloride (108.8 g, 0.8163 mole) in dichloromethane (670 mL), 1,4-bis(2,2,2-trifluoroethoxy) benzene (111.9 g, 0.4081 mole) was added in one portion at 20° C.-25° C. Methyl chlorooxoacetate (100 g, 0.8163 mole) then was added in a thin stream over 5 minutes. The reaction mixture was stirred at 20° C.-25° C. for 3 hours. The reaction mixture was quenched on a mixture of 3 kg of ice and 670 mL of dichloromethane with agitation. Liquid phases were separated and the aqueous layer was washed with dichloromethane (300 mL). The combined organic layers were washed with water (2 L) and concentrated in vacuo to yield crystalline methyl 2,5-bis(2,2,2-trifluoroethoxy) phenylglyoxalate (142.2 g, 96.7% yield).

(2) Preparation of Ethyl 2,5-bis(2,2,2-trifluoroethoxy) Phenylglyoxalate (VIIIb):

To a stirred solution of 1,4-bis(2,2,2-trifluoroethoxy) benzene (III) (500 g, 1.8248 mole) in dichloromethane (5 L) at 20° C.-25° C., anhydrous aluminum chloride (485 g, 3.6373 mole) was added in portions under dry nitrogen atmosphere over 30 minutes. Temperature rose to 28° C. and a dark, very thin slurry was obtained. Ethyl chlorooxoacetate (501.02 g, 3.6697 mole) was added in a thin stream at 20° C.-25° C. over 1.5 hours. The reaction mixture was stirred at 20° C.-25° C. for 7 hours. The reaction mixture was quenched onto a mixture of 4 kg of ice and 1L of water with agitation in 40 minutes. The reaction mixture was stirred at 10° C.-15° C. for 1 hour and then allowed to settle. Liquid phases were separated and the lower organic layer was washed free of acids with water. The organic solution containing ethyl 2,5-bis(2,2,2-trifluoroethoxy) phenylglyoxalate was used for formation of the benzoic acid (II).

(c) Preparation of 2,5-bis(2,2,2-trifluoroethoxy) Benzoic Acid (II)

(1) From Compound VIIIa:

To a stirred solution of methyl 2,5-bis(2,2,2-trifluoroethoxy) phenylglyoxalate (VIIIa) (255 g, 0.7083 mole) in dichloromethane (2.55 L), sodium hydroxide (50% solution in water, 142 mL, 1.7707 mole) and water (459 mL) were added. The mixture was cooled to 0° C.-5° C. Hydrogen peroxide (30% aqueous solution, 67.4 mole, 0.9917 mole) was added in a thin stream at 0° C.-5° C. over 45 minutes. The heterogeneous mixture was warmed and stirred at gentle reflux for 1 hour. The reaction mixture was cooled to 20° C.-25° C., diluted with water (1 L) and stirred for 30 minutes. Phases were separated and the aqueous phase was washed with dichloromethane (1 L). The aqueous alkaline layer was cooled to 0° C.-5° C. and acidified with concentrated. HCl to pH: 0-1. The slurry was stirred at 0°C.-5° C. for 4 hours, filtered and the collected solid was washed with cold (0° C.-5° C.) water (200 mL) and dried. The pure benzoic acid (II) was obtained as an off-white powder (176.5 g, yield 78.4%).

(2) From Compound VIIIb:

To the solution of ethyl 2,5-bis(2,2,2-trifluoroethoxy) phenylglyoxalate (VIIIb), a solution of sodium hydroxide (185 g, 4.62 mole) in 1.085 L of water at 20° C.-25° C. was added. The mixture was cooled to 0°C.-5° C. and 30% aqueous hydrogen peroxide (250 mL, 2.5735 mole) was added in 40 minutes. The reaction mixture was then warmed to 20° C., diluted with 2 L of water, stirred for 1 hour and allowed to settle. The aqueous layer was separated, cooled to 0° C.-5° C., and acidified to pH=1 using concentrated HCl. The white slurry was stirred for 1 hour at 0° C.-5° C. and filtered through a Büchner funnel. The collected solid was washed with cold water (500 mL) and dried to obtain 298 g of 2,5-bis(2,2,2-trifluoroethoxy) benzoic acid (II).

(d) Preparation of 2,5-bis(2,2,2-trifluoroethoxy) Benzoyl Chloride (VII):

To a stirred slurry of 2,5-bis(2,2,2-trifluoroethoxy) benzoic acid (II) (300 g, 0.9434 mole) in toluene (2 L), oxalyl chloride (300 g, 2.3635 mole) was added at 0° C.-5° C. under a dry nitrogen atmosphere during 1 hour. The slurry was heated and stirred at reflux. Within 40 minutes a clear, pale brown solution was obtained. The solution was stirred under reflux for a total of 4 hours. Excess oxalyl chloride and solvent were removed in vacuo. The residual pale brown oil (360 g) was reconstituted in toluene (1 L) and used for the formation of the benzoate (IV).

### Reaction Scheme A, Stage 2b

(d′) Preparation of 2,5-bis(2,2,2 trifluoroethoxy)benzoyl Chloride (VII)

To a slurry of AlCl₃ (0.5053 g, 3.790 mmole) in methylene chloride (5 mL), oxalyl chloride (1.3 mL, 14.59 mmole) was added in one portion. A solution of 1,4-bis(2,2,2-trifluoroethoxy) benzene (m) (0.5057 g, 1.82 mmole) in methylene chloride (17.5 mL) was added dropwise with stirring over 10 minutes at 20° C.-25° C. After 4 hours gas chromatography analysis indicated the presence of 23.0% of unreacted 1,4-bis(2,2,2-trifluoroethoxy) benzene, 42.1% of 2,5-bis(2,2,2-trifluoroethoxy) benzoyl chloride (VII), 2.0% of 2,5-bis(2,2,2-trifluoroethoxy) benzoic acid (II), 16.4% of bis(2,5-bis(2,2,2-trifluoroethoxy) phenyl) ketone (XII), and 16.1% of 2,5-bis(2,2,2-trifluoroethoxy) benzoic anhydride (XI). The benzoyl chloride then is separated by extraction with organic solvents.

(e) Preparation of 2,2,2-trifluoroethyl 2,5-bis(2,2,2-trifluoroethoxy) Benzoate (IV):

To a solution of TFE (188.4 g, 1.88 mole) in toluene (1.5 L) was added triethylamine (114 g). The mixture was cooled to 0°C.-5° C. and the solution of 2,5-bis(2,2,2-trifluoroethoxy) benzoyl chloride (VII) (360 g in 1 L of toluene) prepared by either method described in step (d) or (d′) was added in 1 hour under a dry nitrogen atmosphere. The heterogeneous reaction mixture was warmed and stirred at 20° C.-25° C. for 4 hours. The reaction mixture was diluted with water (2 L), stirred for 30 minutes and the phases were separated. The upper organic phase was washed with water (2 L) and then distilled to remove the solvents. The residual brown oil (365 g) was fractionally distilled in vacuo at 114° C.-115° C. at 0.05 mmHg. Pure 2,2,2-trifluoroethyl 2,5-bis(2,2,2-trifluoroethoxy) benzoate (IV) was collected as a colorless liquid (3.62.7 g, 96.2% yield).

### Reaction Scheme B, Stage 4

(f) Preparation of 2,5-bis(2,2,2-trifluoroethoxy)-N-(2-pyridylmethyl)-benzamide (V):

2,2,2-Trifluoroethyl 2,5-bis(2,2,2-triflouroethoxy) benzoate (50 g, 0.125 mole) in 100 mL glyme was added, over about 40 minutes, to a colorless solution of 2-aminomethylpyridine (16.2 g. 0.15 mole) in 100 mL of glyme. The mixture was stirred under a dry nitrogen atmosphere at about 20 to 25° C. The resulting solution was stirred for about 20 hours at about 20° to 25° C., then stirred with heating to gentle reflux for about 3 hours. The solution was cooled to 20° to 25° C. and then concentrated in vacuo to a volume of 75 mL. Hexane (500 mL) was added with stirring at about 20° to 25° C. in 1 hour. The resulting slurry was stirred at about 20 to 25° C. for 4 hours and then filtered. The collected solids were washed with hexane (100 mL) and the product dried in air at constant weight. The amide (V) was obtained as an off-white powder (50 g, 99% yield), melting point 101-103° C.

### Stage 5

(g) Preparation of Flecainide Acetate (1):

A mixture of the pyridylbenzamide (V) (25 g, 0.06127 mole), glacial acetic acid (200 mL), and platinum oxide catalyst (0.5 g) was shaken in a hydrogen atmosphere at 10 psi for 3 hours. The reaction mixture was filtered to remove the catalyst. The clear dark filtrate was cooled to 5° to 10° C. and treated with 50% aqueous NaOH solution to pH=13-14. The mixture was diluted with water (400 mL) and stirred for 2 hours. The slurry was filtered and the solid was then washed with water until the filtrate was neutral. The solid was dried at 40° C. to constant weight. 2,5-bis(2,2,2-trifluoroethoxy)-N-(2-piperidylmethyl)-benzamide (flecainide free base) was obtained as an off-white powder (24.6 g, m.p. 95°-97° C.).

### Stage 6

To a clean colorless solution of the flecainide free base (24 g, 0.05797 mole) in acetone (240 mL) was added glacial acetic acid (3.65 mL, 0.06377 mole) and the clean solution was stirred at 20° C.-25° C. Within 15 minutes, a precipitate started to separate. The slurry was stirred for a total of 2 hours and then concentrated in vacuo to 96 mL. Hexane (240 mL) was added and the mixture was stirred at 20°-25° C. for 2 hours. The mixture was filtered and the collected solid was washed with hexane (25 mL) and dried. Pure flecainide acetate (I) (25 g, 91% yield) was obtained as a white powder (m.p. 142 to 145° C.).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

It is further to be understood that values are approximate, and are provided for description.

Patents, patent applications, publications, procedures, and the like are cited throughout this application, the disclosures of which are incorporated herein by reference in their entireties.

What is claimed is:

1. A method of preparing flecainide from 1,4-bis(2,2,2-trifluoroethoxy) benzene (III), the improvement comprising producing the (trifluoroethoxy) benzene by reacting 4-fluoro-1-bromobenzene (VI) with 2,2,2-trifluoroethanol (TFE): 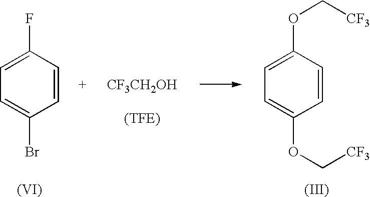
in the presence of a base and a copper-containing catalyst.

2. The method of claim 1, wherein the TFE is reacted in the proportion of from 1 to 3 moles per mole of the 4-fluoro-1 -bromobenzene.

3. A method of preparing flecainide or a pharmaceutically acceptable salt thereof, which comprises:
(a) reacting 4-fluoro-1-bromobenzene (VI) with 2,2,2-trifluoroethanol (TFE) in the presence of base and a copper-containing catalyst to produce 1,4-bis (2,2,2-trifluoroethoxy) benzene (m);
(b) converting the (trifluoroethoxy benzene to 2,5-bis (2,2,2-trifluoroethoxy) benzoate (IV); and
(c) converting the trifluoroethoxy benzoate to flecainide.

4. A method of preparing flecainide or a pharmaceutically acceptable salt thereof, which comprises:
(a) reacting 4-fluoro-1-bromobenzene (VI) with 2,2,2-trifluoroethanol (TFE) int he presence of base and a copper-containing catalyst to produce 1,4-bis (2,2,2-trifluoroethoxy) benzene (III);
(b) converting the (trifluoroethoxy) benzene (III) to 2,5-bis(2,2,2-trifluoroethoxy) benzoyl chloride (VII);
(c) reacting the (trifluoroethoxy) benzoyl chloride with TFE in the presence of a second base to produce 2,5-bis(2,2,2-trifluoroethoxy) benzoate (IV);
(d) reacting the (trifluoroethoxy) benzoate with 2-aminomethylpyridine to produce 2,5-bis(2,2,2-trifluoroethoxy) pyridylbenzamide (V); and
(e) reducing the (trifluoroethoxy) pyridylbenzamide to flecainide.

5. The method of claim 4, wherein the (trifluoroethoxy) benzene (III) is converted to the (trifluoroethoxy) benzoyl chloride (VII) by
(i) acylating the (trifluoroethoxy) benzene with a chlorooxoacetate ROC(O)COCl in the presence of a Lewis acid catalyst, wherein R is C₁-6 alkyl, to form an alkyl 2,5-bis(2,2,2-trifluoroethoxy) phenylglyoxalate (VIII);
(ii) oxidizing the (trifluoroethoxy) phenylglyoxalate to the corresponding benzoic acid (II); and
(iii) reacting the benzoic acid with an acid chloride to form the(trifluoroethoxy) benzoyl chloride (VII).

6. The method of claim 4, wherein the (trifluoroethoxy) benzene (III) is converted to a (trifluoroethoxy) benzoyl chloride (VII) by
(i) reacting the (trifluoroethoxy) benzene with an oxalyl halide in the presence of a Lewis acid catalyst to produce a mixture of the (trifluoroethoxy) benzoyl chloride (VII), bis(2,5-bis(2,2,2-trifluoroethoxy) phenyl ketone (XII), 2,5-bis(2,2,2-trifluoroethoxy) benzoic anhydride (XI) and 2,5-bis(2,2,2-trifluoroethoxy) benzoic acid (II); and
(ii) separating the (trifluoroethoxy) benzoyl chloride from the mixture.

7. The method of claim 4, further comprising:
(f) reacting the flecainide formed in step (e) with acetic acid to produce flecainide acetate;
(g) concentrating the flecainide acetate to produce a concentrated reaction mixture;
(h) diluting the concentrated reaction mixture with an antisolvent for the flecainide acetate; and
(i) isolating the flecainide acetate (I).

8. The method of claim 5, wherein the alkyl 2,5-bis(2,2,2-trifluoroethoxy) phenylglyoxalate (VIII) is methyl 2,5-bis(2,2,2-trifluoroethoxy) phenylglyoxalate (VIIIa).

9. The method of claim 5, wherein the Lewis acid catalyst is aluminum trichloride or boron trifluoride.

10. The method of claim 6, wherein the oxalyl halide is oxalyl chloride or oxalyl bromide, and the Lewis acid catalyst is aluminum trichloride or boron trifluoride.
